(19) United States
(12) Patent Application Publication
(10) Pub. No.: US 2008/0112968 A1
(43) Pub. Date: May 15, 2008

(54) NICOTINAMIDE COMPOSITIONS FOR TREATMENT OF SKIN DISEASES AND DISORDERS

(75) Inventor: Jerzy Gebicki, Lodz (PL)

Correspondence Address: LAHIVE & COCKFIELD, LLP, ONE POST OFFICE SQUARE, BOSTON, MA, 02109-2127, US

(73) Assignee: Dermena, Ottawa (CA)

(21) Appl. No.: 11/870,307

(22) Filed: Oct. 10, 2007

Related U.S. Application Data
(60) Provisional application No. 60/851,275, filed on Oct. 11, 2006.
(60) Provisional application No. 60/852,567, filed on Oct. 18, 2006.

(51) Int. Cl. A61K 36/02; A61K 31/455

(52) U.S. Cl. 424/195.17; 514/355; 514/356

(57) ABSTRACT

The present invention is directed to compositions of nicotinamide derivatives combined with wakame seaweed, wakame extracts, or glycosaminoglycans, and their use in treating skin diseases and disorders.

### RELATED APPLICATIONS

This application claims priority to U.S. Provisional Application No. 60/851,275, Attorney Docket No. PRI-010-1, filed Oct. 11, 2006, titled “Nicotinamide Compositions for Treatment of Skin Diseases and Disorders,” as well as U.S. Provisional Application No. 60/852,567, Attorney Docket No. PRI-010-2, filed Oct. 18, 2006, titled “Nicotinamide Compositions for Treatment of Skin Diseases and Disorders,” which are incorporated herein by reference in their entirety. Additionally, the contents of any patents, patent applications, and references cited throughout this specification are hereby incorporated by reference in their entireties.

### BACKGROUND OF THE INVENTION

Both women and men are constantly seeking ways to maintain a youthful appearance for as long as possible and, consequently, seek to attenuate the signs of skin aging. The first visible signs of aging are usually found on the skin: dryness, fine lines and wrinkles, age spots, red blotches, and sagging and flaccid skin. Dullness and loss of hair are also well-known symptoms. As the skin ages, there is a reduction in protein synthesis, an increase in proteolysis and a general disruption of the skin barrier, connective tissue and skin cohesion. Numerous skin or hair care products are available to consumers for treatment or prevention of these skin conditions that are caused by various external sources of stress, including, for example, atmospheric pollution, mechanical stress, contact with household and other chemicals, as well as sun exposure.

Furthermore, men and women can develop diseases and disorders of the skin that can affect quality of life to a far greater extent than a sign of skin aging. Such diseases and disorders include, but are not limited to: burns, scalds and skin wounds.

Many compounds have been described as being useful for improving skin appearance and physiology, including reducing fine lines, wrinkles and other symptoms associated with aged or photodamaged skin. Also, many compounds and compositions are available for the treatment of more serious skin diseases and disorders. However, a continued need exists to find new therapeutic agents to counteract anti-aging effects as well as treat human skin diseases and disorders.

### SUMMARY OF THE INVENTION

There remains a need for new treatments and therapies for fine lines, wrinkles and other symptoms associated with aged or photodamaged skin. There is also a need for compounds useful in the treatment or prevention or amelioration of one or more symptoms of a skin disease or disorder.

Accordingly, in one aspect, the invention provides a composition comprising wakame seaweed or wakame extract, and a compound of Formula I:

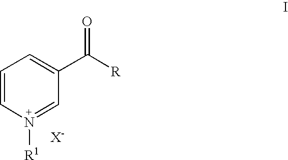

wherein
R represents the group NR²R³, OH, or C₁₋₄-alkyl;
R¹ and R² each, independently, represent hydrogen or C₁₋₄-alkyl;
R³ represents hydrogen, C₁₋₄-alkyl, C₁₋₄-alkyl-OH, or C₁₋₄-alkoxy; and
X⁻ is a physiologically suitable counter-anion.

In one embodiment, the compound of Formula I of the above composition is selected from a 1-methylnicotinamide salt, 1,N′-dimethylnicotinamide salt, 1-methyl-N′,N′-diethylnicotinamide salt, 1-methyl-N′-(hydroxymethyl)-nicotinamide salt, 1-methyl-3-acetylpyridine salt, 1-propylnicotinamide salt, trigonelline salt, and nicotinamide. In another embodiment, the compound of Formula I is a 1-methylnicotinamide salt. In still another embodiment, the compound of Formula I is 1-methylnicotinamide chloride.

In one embodiment, the compound of Formula I is present in said composition in a concentration of between 0.001% and 30% by weight of said composition. In another embodiment, the compound of Formula I is present in said composition in a concentration of between 1% and 20% by weight of said composition. In yet another embodiment, the compound of Formula I is present in said composition in a concentration of between 5% and 15% by weight of said composition. In still another embodiment, the compound of Formula I is present in said composition in a concentration of between 0.2% and 5% by weight of said composition. In another embodiment, the compound of Formula I is present in said composition in a concentration of approximately 1% by weight of said composition.

In another embodiment of the invention, the composition is an anti-wrinkle cream effective for improving the structure of the dermis and restoring firmness and tonicity to the skin of a subject. In another embodiment of the invention, the composition is a topical composition effective for maintaining greasing levels of the skin of a subject. In another embodiment of the invention, the composition is a topical composition effective for maintaining and/or improving the moisture level of the skin of a subject. In another embodiment of the invention, the composition is a topical composition effective for smoothening the skin of a subject.

In another aspect, the invention provides a composition comprising a glycosaminoglycan (GAG) and a compound of Formula I:

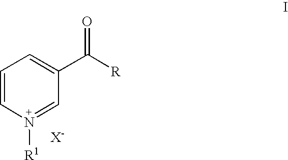

wherein
R represents the group NR²R³, OH, or C₁₋₄-alkyl;
R¹ and R² each, independently, represent hydrogen or C₁₋₄-alkyl;
R³ represents hydrogen, C₁₋₄-alkyl, C₁₋₄-alkyl-OH, or C₁₋₄-alkoxy; and
X⁻ is a physiologically suitable counter-anion.

In one embodiment, the compound of Formula I of the above composition is selected from a 1-methylnicotinamide salt, 1,N′-dimethylnicotinamide salt, 1-methyl-N′,N′-diethylnicotinamide salt, 1-methyl-N′-(hydroxymethyl)-nicotinamide salt, 1-methyl-3-acetylpyridine salt, 1-propylnicotinamide salt, trigonelline salt, and nicotinamide. In another embodiment, the compound of Formula I is a 1-methylnicotinamide salt. In still another embodiment, the compound of Formula I is 1-methylnicotinamide chloride.

In one embodiment, the glycosaminoglycan (GAG) is heparin, heparin sulfate, keratan sulfate, dermatin, dermatin sulfate, heparin-hyaluronic acid, chondroitin, chondroitin sulfate (e.g., chondroitin 6-sulfate and chondroitin 4-sulfate), chitin, chitosan, acetyl-glucosamine, hyaluronic acid, aggrecan, decorin, biglycan, fibromodulin or lumican, or combinations thereof. In a particular embodiment, the GAG is heparin. In another embodiment, the source of the glycosaminoglycan is wakame seaweed or wakame seaweed extract.

In another aspect, the invention provides a method of treating skin diseases and disorders in a subject in need thereof by administering to the subject a composition comprising wakame seaweed or wakame extract, and a compound of Formula I.

In yet another aspect, the invention provides a method of treating skin diseases and disorders in a subject in need thereof by administering to the subject a composition comprising a glycosaminoglycan (GAG) and a compound of Formula I.

In one embodiment, the skin diseases or disorders are selected from the group consisting of sunburn, burns, scalds, skin wounds, wrinkles, oxidative damage in the skin and UV-induced skin damage.

In one aspect, the invention provides a method of restoring firmness and tonicity to the skin in a subject in need thereof by administering to the subject a composition comprising wakame seaweed or wakame extract, and a compound of Formula I.

In another aspect, the invention provides a method of restoring firmness and tonicity to the skin in a subject in need thereof by administering to the subject a composition comprising a glycosaminoglycan (GAG) and a compound of Formula I.

In yet another aspect, the invention provides a method of preventing, retarding, and/or treating wrinkles in a subject in need thereof by administering to the subject a composition comprising wakame seaweed or wakame extract, and a compound of Formula I. In still another aspect, the invention provides a method of preventing, retarding, and/or treating wrinkles in a subject in need thereof by administering to the subject a composition comprising a glycosaminoglycan (GAG) and a compound of Formula I. In one embodiment, the composition is applied periodically for a period of time sufficient to achieve at least a visible reduction of said wrinkle. In another embodiment, the composition is applied on a daily basis. In still another embodiment, the period of time is at least two weeks. In yet another embodiment, the period of time is at least one month. In another embodiment, the period of time is at least two months. In another embodiment, the period of time is at least three months.

In one embodiment, the composition of the invention is formulated in a cream, a balm, an ointment, a liposome formulation, aqueous solution or a gel. In another embodiment, the composition contains an additional component comprising water, glycerine, petrolatum, mineral oil micro-crystalline waxes, paraffins, ozokerite, polyethylene, polybutene, polydecene and perhydrosqualene, dimethicones, cyclomethicones, alkyl siloxanes, polymethylsiloxanes and methylphenylpolysiloxanes, lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate castor oil, soy bean oil, sunflower seed oil, maleated soy bean oil, safflower oil, cotton seed oil, corn oil, walnut oil, peanut oil, olive oil, cod liver oil, almond oil, avocado oil, palm oil or sesame oil, and any combinations thereof. In still another embodiment, the composition contains an additional component comprising polyglyceryl-2-dipolyhydroxystearate, dicocoyl pentaerythrityl distearyl citrate, glycerin, ethylhexyl stearate, dicaprylyl carbonate, cocoglycerides, tocopheryl acetate, DMDM hydantoin, water, vitamin A or vitamin E, and any combinations thereof.

In one embodiment, the composition is administered with the assistance of ultrasound radiation.

In one aspect, the invention provides a composition with anti-aging effects comprising wakame seaweed or wakame extract, and a compound of Formula I. In another aspect, the invention provides a composition with anti-aging effects comprising a glycosaminoglycan (GAG) and a compound of Formula I. In one embodiment, the compound of Formula I is 1-methylnicotinamide chloride.

In another aspect, the composition of the invention comprising wakame seaweed or wakame extract, and a compound of Formula I, is a topical composition. In still another aspect, the composition of the invention comprising a glycosaminoglycan (GAG) and a compound of Formula I is a topical composition.

### BRIEF DESCRIPTION OF THE DRAWINGS

FIG. 1 demonstrates the anti-wrinkle effects of the composition of the invention on a subject over a six-week study.

FIG. 2 demonstrates the anti-wrinkle effects of the composition of the invention on a different subject over a six-week study.

FIG. 3 demonstrates skin smoothness of before use of a composition of the invention.

FIG. 4 demonstrates skin smoothness after four weeks of use of a composition of the invention.

### DETAILED DESCRIPTION OF THE INVENTION

The use of 1-alkylnicotinamide salts for treatment of a wide variety of skin diseases and disorders is described in EP Patent No. 1 147 086 (incorporated herein by reference). Furthermore, wakame seaweed (Undaria pinnatifida) is a common additive in a variety of cosmetics due to its moisturising and anti-aging properties. These beneficial properties of wakame are thought to be attributed to the glycoaminoglycans (GAGs) that are found in the seaweed. GAGs are mucopolysaccharides which can be obtained from numerous sources (e.g., rooster combs, trachea, umbilical cords, skin, articular fluids and certain bacteria such as Streptococci spp). Most glycosaminoglycans (hyaluronic acid, chondroitin sulfates A, B, and C, heparin sulfate, heparin, keratan sulfate, dermatan sulfate, etc.) are composed of repeating sugars such as non-sulfated n-acetylglucosamine, glucuronic acid and n-acetyl galactosamine (these are known as non-sulfated glycosaminoglycans) or polysulfated sugars (sulfated glycosaminoglycans).

The present invention is directed toward compositions comprising 1-alkylnicotinamide salts, e.g., 1-methylnicotinamide salts (MNA), and one or more GAGs, and their use for the treatment of skin diseases and disorders, including, but not limited to, sunburn, burn, scalds, skin wounds, wrinkles, oxidative damage to the skin, UV-induced skin damage, and other effects of aging. Furthermore, the present invention is directed toward compositions comprising 1-alkylnicotinamide salts, e.g., 1-methylnicotinamide salts (MNA) and a wakame extract, and their use for the treatment of skin diseases and disorders, including, but not limited to, sunburn, burn, scalds, skin wounds, wrinkles, oxidative damage to the skin, UV-induced skin damage, and other effects of aging.

### Definitions

These and other embodiments of the invention will be described with reference to following definitions that, for convenience, are collected here.

The language “skin diseases and disorders,” as used herein, describes diseases and disorders that may be treated or prevented (or a symptom of such disease or disorder that may be reduced) by the compounds of the invention. For example, skin diseases and disorders include, but are not limited to, skin diseases and disorders in which oedema, erythema, cutaneous eruption, dilation of superficial blood vessels and desquamation are manifested (including when accompanied by pruritus and burning sensation), as well as in cases of intensified seborrhoea. Skin diseases and disorders also include, but are not limited to, crural ulceration, acne juvenile, acne rosacea, psoriasis, atopic dermatitis and vitiligo. Skin diseases and disorders to be treated by the compositions of the invention also include, but are not limited to, hair loss, especially alopecia areata, androgenic alopecia, and alopecia caused as a side effect of chemotherapy or radiotherapy. Skin diseases and disorders to be treated by the compositions of the invention also include, but are not limited to, burns and scalds (particularly first and first/second degree bums and scalds) and in wound healing, as well as in treating sunburn.

In a particular embodiment, the “skin diseases and disorders” to be treated by the compositions of the invention are selected from the group consisting of sunburn, bums, scalds, skin wounds, wrinkles, oxidative damage in the skin, UV-induced skin damage and any other symptom of the aging process.

The language “wakame extract” is used to refer to any substance, liquid or solid, that is extracted from wakame seaweed. In a particular embodiment, the language “extract” is used to refer to any substance, liquid or solid, that is extracted from wakame seaweed that has a higher concentration, per mg, of one or more GAGs than in the original food source. Such a substance is said to be “enriched” in one or more GAGs. Additionally, the term “extract” refers to either the GAG that is extracted from wakame, or a substance that is extracted from wakame that contains both one or more GAGs as well as other natural products that are derived from the wakame during the extraction process (e.g., an extract containing one or more GAGs, as well as magnesium and other trace minerals that are found in wakame).

Moreover, the term “extract” includes any material resulting from crushing the wakame and mixing with water or other ingredients; chopping, grinding, mincing, or forming a paste of the wakame, or processing the wakame into a dry powder.

In one embodiment, the “wakame extract” is a powder. In another embodiment, the “wakame extract” is a powder that is enriched in one or more GAGs.

As used herein, the term “topical composition” refers to a composition which is suitable for application to the surface of a body part, or a localized area of the body. Preferably, the surface of a body part comprises skin or a mucous membrane. As described herein, a topical composition includes compositions comprising a composition of the invention (e.g., wakame seaweed or wakame extract, and a compound of Formula I, or a GAG and a compound of Formula I) and a cosmetic, including, but not limited to, creams ointments, lotions, gels, solutions or suspensions.

The term “cosmetic” or “cosmetic composition” or “cosmetic product” when used herein means any cosmetic product that can be directly applied to keratinous surfaces such as skin, hair, or nails, including, without limitation, lipstick, mascara, rouge, foundation, blush, eyeliner, lipliner, lip gloss, facial or body powder, sunscreens and blocks, nail polish, mousse, sprays, styling gels, nail conditioner, whether in the form of creams, lotions, gels, ointments, emulsions, colloids, solutions, suspensions, compacts, solids, pencils, spray-on formulations, brush-on formulations and the like. Personal care products that are described by the terms “cosmetic” or “cosmetic composition” or “cosmetic product” include, without limitation, bath and shower gels, shampoos, conditioners, cream rinses, hair dyes and coloring products, leave-on conditioners, sunscreens and sunblocks, lip balms, skin conditioners, hair sprays, soaps, body scrubs, exfoliants, astringents, depilatories and permanent waving solutions, antidandruff formulations, antisweat and antiperspirant compositions, shaving, preshaving and after shaving products, moisturizers, cold creams, deodorants, cleansers, skin gels, rinses, whether in solid, powder, liquid, cream, gel, ointment, lotion, emulsions, colloids, solutions, suspensions, or other form.

The term “keratinous surface” means bodily surfaces such as skin, hair, or nails.

The term “treatment” or “treating,” as used herein, is defined as the application or administration of a therapeutic agent, i.e., a composition of the invention, to a subject, or application or administration of a therapeutic agent to an isolated tissue or cell line from a subject (e.g., for diagnosis or ex vivo applications), who has a skin disease or disorder (e.g., wrinkles), a symptom of a skin disease or disorder or a predisposition toward a skin disease or disorder, with the purpose to cure, heal, alleviate, relieve, alter, remedy, ameliorate, improve or affect the skin disease or disorder, the symptoms of the skin disease or disorder or the skin disease or disorder. Such treatments may be specifically tailored or modified, based on knowledge obtained from the field of pharmacogenomics.

The term “subject” includes living organisms in which skin diseases and disorders can occur, or which are susceptible to skin diseases and disorders. The term “subject” includes animals (e.g., mammals, e.g., cats, dogs, horses, pigs, cows, goats, sheep, rodents, e.g., mice or rats, rabbits, squirrels, bears, primates (e.g., chimpanzees, monkeys, gorillas, and humans)), as well as chickens, ducks, geese, and transgenic species thereof; and cells, e.g., immortalized or nonimmortalized cells, derived therefrom.

Administration of the compositions of the present invention to a subject to be treated can be carried out using known procedures, at dosages and for periods of time effective to inhibit skin diseases and disorders in the subject. An effective amount of the therapeutic compound necessary to achieve a therapeutic effect may vary according to factors such as the state of the disease or disorder in the subject, the age, sex, and weight of the subject, and the ability of the therapeutic compound to inhibit the skin disease or disorder in the subject. Dosage regimens can be adjusted to provide the optimum therapeutic response. For example, several divided doses may be administered daily or the dose may be proportionally reduced as indicated by the exigencies of the therapeutic situation. A non-limiting example of an effective dose range for a composition of the invention (e.g., MNA and wakame extract) is between 1 and 500 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

In particular, the selected dosage level will depend upon a variety of factors including the activity of the particular compound of the present invention employed, the time of administration, the rate of excretion of the particular compound being employed, the duration of the treatment, other drugs, compounds or materials used in combination with the particular compound employed, the age, sex, weight, condition, general health and prior medical history of the patient being treated, and like factors well known in the medical arts.

A medical doctor, e.g., physician or veterinarian, having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

The regimen of administration can affect what constitutes an effective amount. The therapeutic formulations can be administered to the subject either prior to or after the onset of a skin disease or disorder. Further, several divided dosages, as well as staggered dosages, can be administered daily or sequentially, or the dose can be continuously infused, or can be a bolus injection. Further, the dosages of the therapeutic formulations can be proportionally increased or decreased as indicated by the exigencies of the therapeutic or prophylactic situation.

In particular embodiments, it is especially advantageous to formulate compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The specification for the dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a skin disease or disorder in subjects.

### Compositions of the Invention

In one aspect, the compositions of the instant invention comprise wakame seaweed or wakame extract, and a nicotinamide derivative of the Formula I. In another aspect, the compositions of the instant invention comprise a glycosaminoglycan (GAG) and a nicotinamide derivative of the Formula I. In one embodiment, the GAG is heparin, heparin sulfate, keratan sulfate, dermatin, dermatin sulfate, heparin-hyaluronic acid, chondroitin, chondroitin sulfate (e.g., chondroitin 6-sulfate and chondroitin 4-sulfate), chitin, chitosan, acetyl-glucosamine, hyaluronic acid, aggrecan, decorin, biglycan, fibromodulin or lumican, or combinations thereof. In a particular embodiment, the GAG is heparin. In another embodiment, the source of the glycosaminoglycan is wakame seaweed or wakame seaweed extract.

The nicotinamide derivatives of the composition are represented by the Formula I:

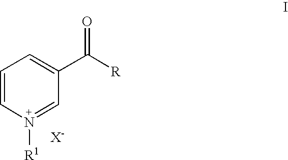

wherein
R represents the group NR²R³, OH, or C₁₋₄-alkyl;
R¹ and R² each, independently, represent hydrogen or C₁₋₄-alkyl;
R³ represents hydrogen, C₁₋₄-alkyl, C₁₋₄-alkyl-OH, or C₁₋₄-alkoxy; and
X⁻ is a physiologically suitable counter-anion.

In one embodiment of Formula I, R represents the group NR²R³. In another embodiment of Formula I, R² represents methyl, ethyl, or hydrogen. In still another embodiment of Formula I, R³ represents CH₂OH, ethyl, or hydrogen. In yet another embodiment of Formula I, R represents OH, NH₂, N(H)CH₃, N(Et)₂, N(H)CH₂OH or CH₃. In another embodiment of Formula I, R¹ represents methyl, ethyl or propyl.

In yet another embodiment, the compound of Formula I is selected from a 1-methylnicotinamide salt or a 1-methyl-N′-hydroxymethylnicotinamide salt. In still another embodiment, the compound of Formula I is selected from a 1,N′-dimethylnicotinamide salt or 1-methyl-N′,N′-diethylnicotinamide salt. In another embodiment, Formula I is selected from a 1-methyl-3-acetylpyridine salt.

In a particular embodiment, the compound of Formula I is selected from the group consisting of 1-methylnicotinamide, 1,N′-dimethylnicotinamide, 1-methyl-N′,N′-diethylnicotinamide, 1-methyl-N′-(hydroxymethyl)-nicotinamide, 1-methyl-3-acetylpyridine, 1-propylnicotinamide, trigonelline, and nicotinamide.

In one embodiment of Formula I, X⁻ is chloride, benzoate, salicylate, acetate, citrate or lactate. In a particular embodiment, X⁻ is chloride. In another embodiment, the compound of Formula I is selected from 1-methylnicotinamide chloride, 1-methylnicotinamide citrate, 1-methylnicotinamide lactate, or 1-methyl-N′-hydroxymethylnicotinamide chloride.

In one embodiment of the composition of the invention, the compound of Formula I is present in said composition in a concentration of between 0.001% and 30% by weight of said composition; in another embodiment the compound of Formula I is present in said composition in a concentration of between 0.01% and 20% by weight of said composition; in another embodiment the compound of Formula I is present in said composition in a concentration of between 0.02% and 5% by weight of said composition; in another embodiment the compound of Formula I is present in said composition in a concentration of less than 1% by weight of said composition. In another embodiment, the compound of Formula I is present in said composition in a concentration of approximately 1% by weight of said composition.

In a preferred embodiment, the invention provides an anti-wrinkle cream effective for restoring firmness and tonicity to the skin of a subject, wherein the anti-wrinkle cream comprises wakame seaweed or wakame extract and a compound of Formula I.

In another preferred embodiment, the invention provides an anti-wrinkle cream effective for restoring firmness and tonicity to the skin of a subject, wherein the anti-wrinkle cream comprises wakame seaweed or wakame extract and a compound of Formula I, wherein the compound of Formula I is 1-methylnicotinamide chloride.

In another preferred embodiment, the invention provides an anti-wrinkle cream effective for restoring firmness and tonicity to the skin of a subject, wherein the anti-wrinkle cream comprises a GAG and a compound of Formula I.

In another preferred embodiment, the invention provides an anti-wrinkle cream effective for restoring firmness and tonicity to the skin of a subject, wherein the anti-wrinkle cream comprises a GAG and a compound of Formula I, wherein the compound of Formula I is 1-methylnicotinamide chloride.

In another preferred embodiment, the invention provides an anti-wrinkle cream effective for restoring firmness and tonicity to the skin of a subject, wherein the anti-wrinkle cream comprises heparin and a compound of Formula I.

In another preferred embodiment, the invention provides an anti-wrinkle cream effective for restoring firmness and tonicity to the skin of a subject, wherein the anti-wrinkle cream comprises heparin and a compound of Formula I, wherein the compound of Formula I is 1-methylnicotinamide chloride.

Without being bound by theory, it is believed that the compositions of the invention are effective in treating skin diseases and disorders (e.g., wrinkles) for the following reasons: experiments have demonstrated that 1-alkylnicotinamide salts, e.g., 1-methylnicotinamide salts (MNA) and the related pyridinium salts can effectively bind GAGs; this binding may be due to formation of complexes based on electrostatic interactions. As described below in the exemplification section, this effect is demonstrated with sepharose immobilized heparin, which represents a model of GAGs. Such binding may facilitate MNA transport into the skin, which leads to the treatment of skin diseases and disorders, e.g., wrinkles. (MNA is a highly hydrophylic molecule with a solubility in water of over 600 g/L.)

Some of the compounds of Formula I are commercially available, for example 1-methylnicotinamide chloride (Sigma) and 1-methylnicotinic acid chloride (Sigma). Alternatively, the compounds can be readily prepared from commercially available compounds (including nicotinamide and nicotinic acid) by synthetic methods well-known to the person skilled in the art. Such methods would include synthesis from appropriately substituted pyridine compounds. Such derivatives are also described in International Patent Application No. PCT/EP2005/050057 and EP Patent No. 1 147 086, both of which are incorporated herein by reference in their entirety.

As used herein, the language “pharmaceutically acceptable salt” or “physiologically suitable counter-anion” refers to a salt of the administered compounds prepared from pharmaceutically acceptable non-toxic acids including inorganic acids, organic acids, solvates, hydrates, or clathrates thereof. Examples of such inorganic acids are hydrochloric, hydrobromic, hydroiodic, nitric, sulfuric, and phosphoric. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. In a particular embodiment, the compound of the invention is in the chloride form of 1-methylnicotinamide.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term “alkyl” also includes alkenyl groups and alkynyl groups. Furthermore, the expression “Cₓ-C_{y}-alkyl”, wherein x is 1-5 and y is 2-10 indicates a particular alkyl group (straight- or branched-chain) of a particular range of carbons. For example, the expression C₁-C₄-alkyl includes, but is not limited to, methyl, ethyl, propyl, butyl, isopropyl, tert-butyl and isobutyl. Moreover, the term C₃₋₆-cycloalkyl includes, but is not limited to, cyclopropyl, cyclopentyl, and cyclohexyl. As discussed below, these alkyl groups, as well as cycloalkyl groups, may be further substituted.

The term alkyl further includes alkyl groups which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀ for straight chain, C₃-C₁₀ for branched chain), and more preferably 6 or fewer carbons. Likewise, preferred cycloalkyls have from 4-7 carbon atoms in their ring structure, and more preferably have 5 or 6 carbons in the ring structure.

Moreover, alkyl (e.g., methyl, ethyl, propyl, butyl, pentyl, hexyl, etc.) include both “unsubstituted alkyl” and “substituted alkyl”, the latter of which refers to alkyl moieties having substituents replacing a hydrogen on one or more carbons of the hydrocarbon backbone, which allow the molecule to perform its intended function.

The term “substituted” is intended to describe moieties having substituents replacing a hydrogen on one or more atoms, e.g. C, O or N, of a molecule. Such substituents can include, for example, oxo, alkyl, alkoxy, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, amino (including alkyl amino, dialkylamino, arylamino, diarylamino, and alkylarylamino), acylamino (including alkylcarbonylamino, arylcarbonylamino, carbamoyl and ureido), dialkylaminocarbonyl, alkylthiocarbonyl, alkoxyl, phosphate, phosphonato, phosphinato, amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, morpholino, phenol, benzyl, phenyl, piperizine, cyclopentane, cyclohexane, pyridine, 5H-tetrazole, triazole, piperidine, or an aromatic or heteroaromatic moiety, and any combination thereof.

Further examples of substituents of the invention, which are not intended to be limiting, include moieties selected from straight or branched alkyl (preferably C₁-C₅), cycloalkyl (preferably C₃-C₈), alkoxy (preferably C₁-C₆), thioalkyl (preferably C₁-C₆), alkenyl (preferably C₂-C₆), alkynyl (preferably C₂-C₆), heterocyclic, carbocyclic, aryl (e.g., phenyl), aryloxy (e.g., phenoxy), aralkyl (e.g., benzyl), aryloxyalkyl (e.g., phenyloxyalkyl), arylacetamidoyl, alkylaryl, heteroaralkyl, alkylcarbonyl and arylcarbonyl or other such acyl group, heteroarylcarbonyl, or heteroaryl group, (CR′R″)₀₋₃NR′R″ (e.g., —NH₂), (CR′R″)₀₋₃CN (e.g., —CN), —NO₂, halogen (e.g., —F, —Cl, —Br, or —I), (CR′R″)₀₋₃C(halogen)₃ (e.g., —CF₃), (CR′R″)₀₋₃CH(halogen)₂, (CR′R″)₀₋₃CH₂(halogen), (CR′R″)₀₋₃CONR′R″, (CR′R″)₀₋₃(CNH)NR′R″, (CR′R″)₀₋₃S(O)₁₋₂NR′R″, (CR′R″)₀₋₃CHO, (CR′R″)₀₋₃O(CR′R″)₀₋₃H, (CR′R″)₀₋₃S(O)₀₋₃R′ (e.g., —SO₃H, —OSO₃H), (CR′R″)₀₋₃O(CR′R″)₀₋₃H (e.g., —CH₂OCH₃ and —OCH₃), (CR′R″)₀₋₃S(CR′R″)₀₋₃H (e.g., —SH and —SCH₃), (CR′R″)₀₋₃OH (e.g., —OH), (CR′R″)₀₋₃COR′, (CR′R″)₀₋₃(substituted or unsubstituted phenyl), (CR′R″)₀₋₃(C₃-C₈ cycloalkyl), (CR′R″)₀₋₃CO₂R′ (e.g., —CO₂H), or (CR′R″)₀₋₃OR′ group, or the side chain of any naturally occurring amino acid; wherein R′ and R″ are each independently hydrogen, a C₁-C₅ alkyl, C₂-C₅ alkenyl, C₂-C₅ alkynyl, or aryl group. Such substituents can include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, oxime, sulfhydryl, alkylthio, arylthio, thiocarboxylate, sulfates, sulfonato, sulfamoyl, sulfonamido, nitro, trifluoromethyl, cyano, azido, heterocyclyl, or an aromatic or heteroaromatic moiety, and any combination thereof. In certain embodiments, a carbonyl moiety (C═O) may be further derivatized with an oxime moiety, e.g., an aldehyde moiety may be derivatized as its oxime (—C═N—OH) analog. It will be understood by those skilled in the art that the moieties substituted on the hydrocarbon chain can themselves be substituted, if appropriate. Cycloalkyls can be further substituted, e.g., with the substituents described above. An “aralkyl” moiety is an alkyl substituted with an aryl (e.g., phenylmethyl (i.e., benzyl)).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

### Combination Therapies

The nicotinamide derivatives of the present invention are intended to be useful, e.g., in the methods of present invention, in combination with one or more additional compounds useful for treating skin diseases and disorders. These additional compounds may comprise compounds of the present invention or compounds, e.g., commercially available compounds, known to treat, prevent, or reduce the symptoms of a skin disease or disorder.

In particular, the nicotinamide derivatives of Formula I can be co-administered with wakame seaweed, a wakame extract, and/or a GAG. Examples of GAGs useful for purposes of the invention include, but are not limited to, heparin, heparin sulfate, keratan sulfate, dermatin, dermatin sulfate, heparin-hyaluronic acid, chondroitin, chondroitin sulfate (e.g., chondroitin 6-sulfate and chondroitin 4-sulfate), chitin, chitosan, acetyl-glucosamine, hyaluronic acid, aggrecan, decorin, biglycan, fibromodulin or lumican, or combinations thereof.

In a particular embodiment, the composition of the invention comprises approximately a 1:9, 2:8, 3:7, 4:6, 5:5, 6:4, 7:3, 8:2 or 9:1 by weight ratio of a compound of Formula I and wakame seaweed or wakame extract.

In a preferred embodiment, the composition of the invention comprises approximately a 1:9, 2:8, 3:7, 4:6 or 5:5 by weight ratio of a compound of Formula I and wakame seaweed or wakame extract.

In another preferred embodiment, the composition of the invention comprises approximately a 1:9, 2:8, 3:7, 4:6 or 5:5 by weight ratio of 1-methylnicotinamide chloride and wakame extract.

In still another preferred embodiment, the composition of the invention comprises approximately a 1:9, 2:8 or 3:7 by weight ratio of 1-methylnicotinamide chloride and wakame extract.

In yet another preferred embodiment, the composition of the invention comprises approximately a 1:9 by weight ratio of 1-methylnicotinamide chloride and wakame extract.

In accordance with an aspect of the present invention there is provided a method of achieving a therapeutic effect for treating a patient suffering from a skin disease or disorder comprising administering a therapeutically effective amount of (i) a pharmaceutical combination comprising as active ingredients a nicotinamide of Formula I, and wakame seaweed or wakame extract to the patient, or (ii) a pharmaceutical combination comprising as active ingredients a nicotinamide of Formula I, and a GAG to the patient. In another embodiment of this aspect of the present invention the therapeutic effect achieved is synergistic, in that, the therapeutic effect is greater than the sum of the therapeutic effect achieved by the administration of the active ingredients separately.

In another embodiment, the composition of the invention consists of a nicotinamide derivative of Formula I, wakame seaweed or extract, and a GAG

In some embodiments, a nicotinamide derivative of Formula I and the wakame seaweed, wakame extract or GAG are included in a single composition, which is administered to a subject having a skin disease or disorder. In other embodiments, a nicotinamide derivative of Formula I and the wakame seaweed, wakame extract or GAG are administered separately to such a subject. The first and at least one second compound may either be co-administered to a subject (i.e., at the same time) or be administered sequentially (i.e., one after the other).

A combination of compounds described herein can either result in synergistic increase in effectiveness against a skin disease or disorder (e.g., wrinkles), relative to effectiveness following administration of each compound when used alone, or such an increase can be additive. Compositions described herein typically include lower dosages of each compound in a composition, thereby avoiding adverse interactions between compounds and/or harmful side effects, such as ones which have been reported for similar compounds. Furthermore, normal amounts of each compound when given in combination could provide for greater efficacy in subjects who are either unresponsive or minimally responsive to each compound when used alone.

A synergistic effect can be calculated, for example, using suitable methods such as, for example, the Sigmoid-Emax equation (Holford, N. H. G. and Scheiner, L. B., Clin. Pharmacokinet. 6:429-453 (1981)), the equation of Loewe additivity (Loewe, S. and Muischnek, H., Arch. Exp. Pathol Pharmacol. 114:313-326 (1926)) and the median-effect equation (Chou, T. C. and Talalay, P., Adv. Enzyme Regul. 22:27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

Nicotinamide derivatives of the invention (i.e., the compounds of Formula I) for administration can be in the range of from about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg. The nicotinamide derivatives of the invention may be administered in combination with wakame seaweed, wakame extract or GAG, wherein the wakame seaweed, wakame extract or GAG is administered in a ranges described above.

In some embodiments, the dose of a nicotinamide derivative of the invention is between about 0.0001 mg and about 25 mg. In some embodiments, a dose of a nicotinamide derivative of the invention used in compositions described herein is less than about 100 mg, or less than about 80 mg, or less than about 60 mg, or less than about 50 mg, or less than about 30 mg, or less than about 20 mg, or less than about 10 mg, or less than about 5 mg, or less than about 2 mg, or less than about 0.5 mg. Similarly, in some embodiments, a dose of a second compound (i.e., wakame seaweed, wakame extract or GAG) as described herein is less than about 1000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg.

### Formulations for Administration

In another embodiment, the present invention is directed to a packaged pharmaceutical composition comprising a container holding a therapeutically effective amount of a nicotinamide derivative of Formula I and wakame seaweed, wakame extract or GAG, and instructions for using the composition to treat, prevent, or reduce one or more symptoms of one or more skin diseases or disorders in a subject.

The term “container” includes any receptacle for holding the pharmaceutical composition. For example, in one embodiment, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions can contain information pertaining to the compound's ability to perform its intended function, e.g., treating, preventing, or reducing one or more skin diseases or disorders in a subject.

Another embodiment of the invention is a pharmaceutical composition comprising a therapeutically effective amount of a composition of the invention and a pharmaceutically acceptable carrier.

The language “therapeutically effective amount” describes the amount of nicotinamide derivative of Formula I of the invention that is effective to treat one or more skin diseases or disorders in a subject.

The language “pharmaceutically acceptable carrier” includes a pharmaceutically acceptable material, composition or carrier, such as a liquid or solid filler, diluent, excipient, solvent or encapsulating material, involved in carrying or transporting a compound(s) of the present invention within or to the subject such that it can perform its intended function. Typically, such compounds are carried or transported from one organ, or portion of the body, to another organ, or portion of the body. Each carrier must be “acceptable” in the sense of being compatible with the other ingredients of the formulation, and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. As used herein “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds can also be incorporated into the compositions.

The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin. In one embodiment, the pharmaceutically acceptable carrier is not DMSO alone.

The compounds for use in the invention can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Topical Formulations

In a particularly preferred embodiment, the compositions of the invention are administered to a subject in a topical formulation. The topical compositions useful in the present invention may be made into a wide variety of product forms such as are known in the art. These include, but are not limited to, cosmetic and cosmetic compositions, as well as lotions, creams, gels, sticks, shampoos, soaps, sprays, ointments, pastes and mousses. These product forms may comprise several types of carriers including, but not limited to, solutions, aerosols, emulsions, gels, solids, and liposomes. Topical formulations are most suitably in the form of an ointment, gel, cream, shampoo, soap, spray, lotion or a solution.

Preferred is topical administration to the skin at the location of the principal manifestation of the skin disease or disorder, (e.g., wrinkle, bum or other skin wound).

The topical formulations of the present invention comprise a safe and effective amount of a dermatologically acceptable carrier within which the compositions of the invention are incorporated to enable the compound of Formula I and the additional components to be delivered to the skin or other relevant site at an appropriate concentration. The carrier can thus act as a diluent, dispersant, solvent, or the like which ensures that the formulation can be applied to and distributed evenly over the selected target to provide an appropriate concentration of the composition of the invention.

Preferred topical formulations according to the present invention comprise about 90 to 99.95% of a pharmaceutical base carrier and about 0.005 to about 10% by weight of a composition of Formula I as defined above and wakame seaweed, wakame extract or GAG. More preferably the topical formulation contains about 0.1 to about 5% by weight of a composition of Formula I as defined above and wakame seaweed, wakame extract or GAG. Preferred pharmaceutical base carriers are an ointment, gel, or aqueous solution.

In an ointment the composition of Formula I as defined above and wakame seaweed, wakame extract or GAG is preferably present at a concentration by weight of 0.1 to 10%, more preferably 0.2 to 5%. In a gel the composition of Formula I as defined above and wakame seaweed, wakame extract or GAG is preferably present in a concentration by weight of 0.05 to 2%, more preferably 0.05 to 1%. In a solution, the composition of Formula I as defined above and wakame seaweed, wakame extract or GAG is preferably present in a concentration by weight of 0.005 to 0.1%, more preferably 0.005 to 0.05%, most preferably 0.01%.

The carrier may contain one or more dermatologically acceptable solid, semi-solid or liquid fillers, diluents, solvents, extenders and the like. The carrier may be solid, semi-solid or liquid. Preferred carriers are substantially liquid. The carrier can itself be inert or it can possess dermatological benefits of its own. Concentrations of the carrier can vary with the carrier selected and the intended concentrations of the compound of Formula I and the other optional components.

Suitable carriers for topical formulations include conventional or otherwise known carriers that are dermatologically acceptable. The carrier should also be physically and chemically compatible with the composition of the invention, and should not unduly impair stability, efficacy or other benefits associated with the formulations of the present invention. Preferred components of the formulations of the present invention should be capable of being comingled in a manner such that there is no interaction which would substantially reduce the efficacy of the formulation under ordinary use situations.

Preferred carriers contain a dermatologically acceptable, hydrophilic diluent. As used herein, “diluent” includes materials in which the composition of the invention can be dispersed, dissolved, or otherwise incorporated. Nonlimiting examples of hydrophilic diluents are water, organic hydrophilic diluents such as lower monovalent alcohols (e.g., C₁-C₄) and low molecular weight glycols and polyols, including propylene glycol, polyethylene glycol (e.g., Molecular Weight 200-600 g/mole), polypropylene glycol (e.g., Molecular Weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof. Water is a preferred diluent. The composition preferably comprises from about 60% to about 99.99% of the hydrophilic diluent

Solutions according to the subject invention typically include a dermatologically acceptable hydrophilic diluent. Solutions useful in the subject invention preferably contain from about 60% to about 99.99% of the hydrophilic diluent.

Aerosols according to the subject invention can be formed by adding a propellant to a solution such as described above. Exemplary propellants include chloro-fluorinated lower molecular weight hydrocarbons. Additional propellants that are useful herein are described in Sagarin, Cosmetics Science and Technology, 2nd Edition, Vol. 2, pp. 443-465 (1972), incorporated herein by reference. Aerosols are typically applied to the skin as a spray-on product

The topical compositions of the subject invention, including, but not limited to, lotions and creams, may comprise a dermatologically acceptable emollient. Such compositions preferably contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics Science and Technology, 2nd Edition, Vol. 1, pp. 32-43 (1972), incorporated herein by reference, contains numerous examples of materials suitable as an emollient

Lotions and creams according to the present invention generally comprise a solution carrier system and one or more emollients. Lotions typically comprise from about 1% to about 20%, preferably from about 5% to about 10%, of emollient; from about 50% to about 90%, preferably from about 60% to about 80%, water. A cream typically comprises from about 5% to about 50%, preferably from about 10% to about 20%, of emollient; and from about 45% to about 85%, preferably from about 50% to about 75%, water.

Ointments of the present invention may comprise a simple carrier base of animal or vegetable oils or semi-solid hydro-carbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology, 2nd edition, Vol. 1, pp. 72-73 (1972), incorporated herein by reference, and/or an emollient. For example, an ointment may comprise from about 2% to about 10% of an emollient; and from about 0.1% to about 2% of a thickening agent.

Preferred ointments comprise Eucerine and glycerol; preferred gels comprise methylcellulose, glycerol and water, or comprise polyacrylic acid, polyethylene glycol, ethanol, triethanolamine, paraben and water; preferred solutions comprise aqueous solutions or solutions of ethyl alcohol or propylene glycol.

Carriers for topical formulations of the compositions of the invention may also include one or more vitamins, such as vitamin A or vitamin E.

Preferred carriers for topical formulations of the compositions of the invention include one or more of the following: polyglyceryl-2-dipolyhydroxystearate, dicaprylyl ether, cocoglycerides, cera alba, sorbitan sesquioleate, aluminium stearates, dicocoyl pentaerythrityl distearyl citrate, dicocoyl pentaerythrityl distearyl citrate, sorbitan sesquioleate, glycerin, ethylhexyl stearate, dicaprylyl carbonate, cocoglycerides, tocopheryl acetate, DMDM hydantoin, methylparaben, phenoxyethanol, propylparaben, vitamin A, vitamin E, and water.

### Oral Administration

For example, for oral administration the compounds can be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets can be coated using suitable methods and coating materials such as OPADRY™ film coating systems available from Colorcon, West Point, Pa. (e.g., OPADRY™ OY Type, OY-C Type, Organic Enteric OY-P Type, Aqueous Enteric OY-A Type, OY-PM Type and OPADRY™ White, 32K18400). Liquid preparation for oral administration can be in the form of solutions, syrups or suspensions. The liquid preparations can be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

### Parenteral Administration

For parenteral administration, the compounds for use in the method of the invention can be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents can be used.

### Transmucosal Administration

Transmucosal administration is carried out using any type of formulation or dosage unit suitable for application to mucosal tissue. For example, the selected active agent can be administered to the buccal mucosa in an adhesive tablet or patch, sublingually administered by placing a solid dosage form under the tongue, lingually administered by placing a solid dosage form on the tongue, administered nasally as droplets or a nasal spray, administered by inhalation of an aerosol formulation, a non-aerosol liquid formulation, or a dry powder, placed within or near the rectum (“transrectal” formulations), or administered to the urethra as a suppository, ointment, or the like.

### Transurethal Administration

With regard to transurethal administration, the formulation can comprise a urethral dosage form containing the active agent and one or more selected carriers or excipients, such as water, silicone, waxes, petroleum jelly, polyethylene glycol (“PEG”), propylene glycol (“PG”), liposomes, sugars such as mannitol and lactose, and/or a variety of other materials. A transurethral permeation enhancer can be included in the dosage from. Examples of suitable permeation enhancers include dimethylsulfoxide (“DMSO”), dimethyl formamide (“DMF”), N,N-dimethylacetamide (“DMA”), decylmethylsulfoxide (“C10 MSO”), polyethylene glycol monolaurate (“PEGML”), glycerol monolaurate, lecithin, the 1-substituted azacycloheptan-2-ones, particularly 1-n-dodecylcyclazacycloheptan-2-one (available under the trademark Azone™ from Nelson Research & Development Co., Irvine, Calif.), SEPA™ (available from Macrochem Co., Lexington, Mass.), surfactants as discussed above, including, for example, Tergitol™, Nonoxynol-9™ and TWEEN-80™, and lower alkanols such as ethanol.

### Transrectal Administration

Transrectal dosage forms may include rectal suppositories, creams, ointments, and liquid formulations (enemas). The suppository, cream, ointment or liquid formulation for transrectal delivery comprises a therapeutically effective amount of the selected active agent and one or more conventional nontoxic carriers suitable for transrectal drug administration. The transrectal dosage forms of the present invention can be manufactured using conventional processes. The transrectal dosage unit can be fabricated to disintegrate rapidly or over a period of several hours. The time period for complete disintegration may be in the range of from about 10 minutes to about 6 hours, e.g., less than about 3 hours.

### Vaginal or Perivaginal Administration

Vaginal or perivaginal dosage forms may include vaginal suppositories, creams, ointments, liquid formulations, pessaries, tampons, gels, pastes, foams or sprays. The suppository, cream, ointment, liquid formulation, pessary, tampon, gel, paste, foam or spray for vaginal or perivaginal delivery comprises a therapeutically effective amount of the selected active agent and one or more conventional nontoxic carriers suitable for vaginal or perivaginal drug administration. The vaginal or perivaginal forms of the present invention can be manufactured using conventional processes as disclosed in Remington: The Science and Practice of Pharmacy, supra (see also drug formulations as adapted in U.S. Pat. Nos. 6,515,198; 6,500,822; 6,417,186; 6,416,779; 6,376,500; 6,355,641; 6,258,819; 6,172,062; and 6,086,909). The vaginal or perivaginal dosage unit can be fabricated to disintegrate rapidly or over a period of several hours. The time period for complete disintegration may be in the range of from about 10 minutes to about 6 hours, e.g., less than about 3 hours.

### Intranasal or Inhalation Administration

The active agents may also be administered intranasally or by inhalation. Compositions for intranasal administration are generally liquid formulations for administration as a spray or in the form of drops, although powder formulations for intranasal administration, e.g., insufflations, nasal gels, creams, pastes or ointments or other suitable formulators can be used. For liquid formulations, the active agent can be formulated into a solution, e.g., water or isotonic saline, buffered or unbuffered, or as a suspension. In certain embodiments, such solutions or suspensions are isotonic relative to nasal secretions and of about the same pH, ranging e.g., from about pH 4.0 to about pH 7.4 or, from about pH 6.0 to about pH 7.0. Buffers should be physiologically compatible and include, for example, phosphate buffers. Furthermore, various devices are available in the art for the generation of drops, droplets and sprays, including droppers, squeeze bottles, and manually and electrically powered intranasal pump dispensers. Active agent containing intranasal carriers can also include nasal gels, creams, pastes or ointments with a viscosity of, e.g., from about 10 to about 6500 cps, or greater, depending on the desired sustained contact with the nasal mucosal surfaces. Such carrier viscous formulations may be based upon, for example, alkylcelluloses and/or other biocompatible carriers of high viscosity well known to the art (see e.g., Remington: The Science and Practice of Pharmacy, supra). Other ingredients, such as preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odor for the formulation. Formulations for inhalation may be prepared as an aerosol, either a solution aerosol in which the active agent is solubilized in a carrier (e.g., propellant) or a dispersion aerosol in which the active agent is suspended or dispersed throughout a carrier and an optional solvent. Non-aerosol formulations for inhalation can take the form of a liquid, typically an aqueous suspension, although aqueous solutions may be used as well. In such a case, the carrier is typically a sodium chloride solution having a concentration such that the formulation is isotonic relative to normal body fluid. In addition to the carrier, the liquid formulations can contain water and/or excipients including an antimicrobial preservative (e.g., benzalkonium chloride, benzethonium chloride, chlorobutanol, phenylethyl alcohol, thimerosal and combinations thereof), a buffering agent (e.g., citric acid, potassium metaphosphate, potassium phosphate, sodium acetate, sodium citrate, and combinations thereof), a surfactant (e.g., polysorbate 80, sodium lauryl sulfate, sorbitan monopalmitate and combinations thereof), and/or a suspending agent (e.g., agar, bentonite, microcrystalline cellulose, sodium carboxymethylcellulose, hydroxypropyl methylcellulose, tragacanth, veegum and combinations thereof). Non-aerosol formulations for inhalation can also comprise dry powder formulations, particularly insufflations in which the powder has an average particle size of from about 0.1 μm to about 50 μm, e.g., from about 1 μm to about 25 μm.

### Transdermal Administration

The compounds of the invention may also be administered through the skin or mucosal tissue using conventional transdermal drug delivery systems, wherein the agent is contained within a laminated structure (typically referred to as a transdermal “patch”) that serves as a drug delivery device to be affixed to the skin. Transdermal drug delivery may involve passive diffusion or it may be facilitated using electrotransport, e.g., iontophoresis. In a typical transdermal “patch,” the drug composition is contained in a layer, or “reservoir,” underlying an upper backing layer. The laminated structure may contain a single reservoir, or it may contain multiple reservoirs. In one type of patch, referred to as a “monolithic” system, the reservoir is comprised of a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are separate and distinct layers, with the adhesive underlying the reservoir which, in this case, may be either a polymeric matrix as described above, or it may be a liquid or hydrogel reservoir, or may take some other form.

### Ultrasound Administration

The administration of the compositions of the invention to a subject may be ultrasound assisted. The term “ultrasound assisted,” as used herein, generally refers to the delivery of compositions of the invention (charged, uncharged, or mixtures thereof), through a body surface (such as skin, mucous membrane, or nails) wherein the delivery is at least partially induced or aided by the application of ultrasonic energy in the form(s) of high frequency sound waves and/or vibrations. As used herein, the term “ultrasound” or “ultrasound energy” is a broad term and is used in its ordinary sense and means, without limitation, mechanical energy transferred through pressure or compression waves with a frequency greater than about 20 KHz. In one embodiment, the waves of the ultrasound energy have a frequency between about 500 KHz and 20 MHz and in another embodiment between about 1 MHz and 3 MHz. In yet another embodiment, the waves of the ultrasound energy have a frequency of about 3 MHz. The term “ultrasound” includes diagnostic, therapeutic and focused ultrasound. Diagnostic ultrasound refers to an ultrasound energy source in a range up to about 100 mW/cm² (FDA recommendation). Therapeutic ultrasound refers to an ultrasound energy source in a range up to about 3-4 W/cm² (WHO recommendation).

Focused ultrasound (FUS) allows thermal energy to be delivered without an invasive probe (see Morocz et al. 1998 Journal of Magnetic Resonance Imaging Vol. 8, No. 1, pp. 136-142). Another form of focused ultrasound is high intensity focused ultrasound (HIFU) which is reviewed by Moussatov et al. in Ultrasonics 1998 Vol. 36, No. 8, pp. 893-900 and TranHuuHue et al. in Acustica, 1997, Vol. 83, No. 6, pp. 1103-1106.

In a particular embodiment, the compositions of the invention are administered to a subject using “ultrasound assistance” from the U-Strip transdermal delivery system, A-wand antiseptic delivery system, and/or U-wand cosmetic delivery system as provided by Dermisonics (http://www.dermisonics.com/).

### Iontophoresis

The administration of the compositions of the invention to a subject may also be iontophoresis assisted. The term “iontophoresis,” as used herein, refers generally to the delivery of a therapeutic agent (charged, uncharged, or mixtures thereof) through a body surface (such as skin, mucous membrane, or nails) wherein the delivery is at least partially induced or aided by the application of an electric potential. As is known in the art, iontophoresis, an electrotransport process, involves the electrically induced transport of charged ions.

In many instances, more than one of the noted processes may be occurring simultaneously to different extents. Accordingly, the term “iontophoresis” is given herein its broadest possible interpretation, to include the electrically induced or enhanced transport of at least one charged or uncharged agent, or mixtures thereof (e.g., a compound of Formula I and wakame extract), regardless of the specific mechanism(s) by which the agent is actually being transported.

In typical transdermal iontophoresis system a low constant current, ranging from micro-Amps to several milli-Amps, is applied for prolonged periods of time ranging from minutes to days. Alternatively, low constant voltage, ranging from milli volts to several volts is applied for prolonged periods of time ranging from minutes to days. The target amperage or voltage may also be achieved by a slow ramping up of the applied electric condition. Alternatively, starting from the target amperage or voltage, the electrical conditions may also be ramped down over time. Alternatively, consecutive pulses using the above electrical conditions are applied during the total duration of iontophoresis. Collectively, the above electrical conditions are referred to herein as “iontophoresis energy”. The above conditions are different from the electrical conditions as applied in the field of electroporation and do not result in measurable pore formation through cell membrane.

Devices that deliver active substances using iontophoresis have been developed for many applications, most of which involve the delivery of pharmaceutical compounds through the subject's skin and into the circulatory system or other organs of a subject's body. Devices for the facilitation of the administration of the compositions of the invention to a subject using iontophoresis are known to those of skill in the art.

### Intrathecal Administration

One common system utilized for intrathecal administration is the APT Intrathecal treatment system available from Medtronic, Inc. APT Intrathecal uses a small pump that is surgically placed under the skin of the abdomen to deliver medication directly into the intrathecal space. The medication is delivered through a small tube called a catheter that is also surgically placed. The medication can then be administered directly to cells in the spinal cord involved in conveying sensory and motor signals associated with lower urinary tract disorders.

### Intravesical Administration

The term intravesical administration is used herein in its conventional sense to mean delivery of a drug directly into the bladder. Suitable methods for intravesical administration can be found, for example, in U.S. Pat. Nos. 6,207,180 and 6,039,967.

### Additional Administration Forms

Additional dosage forms of this invention include dosage forms as described in U.S. Pat. No. 6,340,475, U.S. Pat. No. 6,488,962, U.S. Pat. No. 6,451,808, U.S. Pat. No. 5,972,389, U.S. Pat. No. 5,582,837, and U.S. Pat. No. 5,007,790. Additional dosage forms of this invention also include dosage forms as described in U.S. patent application Ser. No. 20030147952, U.S. patent application Ser. No. 20030104062, U.S. patent application Ser. No. 20030104053, U.S. patent application Ser. No. 20030044466, U.S. patent Application Ser. No. 20030039688, and U.S. patent application Ser. No. 20020051820. Additional dosage forms of this invention also include dosage forms as described in PCT Patent Application WO 03/35041, PCT Patent Application WO 03/35040, PCT Patent Application WO 03/35029, PCT Patent Application WO 03/35177, PCT Patent Application WO 03/35039, PCT Patent Application WO 02/96404, PCT Patent Application WO 02/32416, PCT Patent Application WO 01/97783, PCT Patent Application WO 01/56544, PCT Patent Application WO 01/32217, PCT Patent Application WO 98/55107, PCT Patent Application WO 98/11879, PCT Patent Application WO 97/47285, PCT Patent Application WO 93/18755, and PCT Patent Application WO 90/11757.

### Controlled Release Formulations and Drug Delivery Systems

In certain embodiments, the formulations of the present invention can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time can be as long as a month or more and should be a release which is longer that the same amount of agent administered in bolus form.

For sustained release, the compounds can be formulated with a suitable polymer or hydrophobic material which provides sustained release properties to the compounds. As such, the compounds for use the method of the invention can be administered in the form of microparticles for example, by injection or in the form of wafers or discs by implantation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes after drug administration.

### Dosing

The therapeutically effective amount or dose of a compound of the present invention will depend on the age, sex and weight of the patient, the current medical condition of the patient and the nature of skin diseases or disorders being treated. The skilled artisan will be able to determine appropriate dosages depending on these and other factors.

A suitable dose of a compound of the present invention can be in the range of from about 0.001 mg to about 500 mg per day, such as from about 0.01 mg to about 100 mg, for example, from about 0.05 mg to about 50 mg, such as about 0.5 mg to about 25 mg per day. The dose can be administered in a single dosage or in multiple dosages, for example from 1 to 4 or more times per day. When multiple dosages are used, the amount of each dosage can be the same or different. For example a dose of 1 mg per day can be administered as two 0.5 mg doses, with about a 12 hour interval between doses.

It is understood that the amount of compound dosed per day can be administered every day, every other day, every 2 days, every 3 days, every 4 days, every 5 days, etc. For example, with every other day administration, a 5 mg per day dose can be initiated on Monday with a first subsequent 5 mg per day dose administered on Wednesday, a second subsequent 5 mg per day dose administered on Friday, etc.

The compounds for use in the method of the invention can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

### Equivalents

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including reaction times, reaction size/volume, and experimental reagents, such as solvents, catalysts, pressures, atmospheric conditions, e.g., nitrogen atmosphere, and reducing/oxidizing agents, etc., with art-recognized alternatives and using no more than routine experimentation, are within the scope of the present application.

It is to be understood that wherever values and ranges are provided herein, e.g., in ages of subject populations, dosages, and blood levels, all values and ranges encompassed by these values and ranges, are meant to be encompassed within the scope of the present invention. Moreover, all values that fall within these ranges, as well as the upper or lower limits of a range of values, are also contemplated by the present application.

### Incorporation by Reference

The contents of all references, issued patents, and published patent applications cited throughout this application are hereby expressly incorporated by reference in their entireties. It should be understood that the use of any of the compounds described herein are within the scope of the present invention and are intended to be encompassed by the present invention and are expressly incorporated herein for all purposes.

### Exemplification of the Invention

The invention is further illustrated by the following examples, which should not be construed as further limiting.

### EXAMPLE 1

### Preparation of Wakame Extract

Dried seaweed (100 g) was powdered in a coffee grinder and suspended in 500 mL of water—96% ethanol solution (2:1, v/v). The mixture was vigorously stirred for 2 hours at room temperature, then filtered through a paper filter. The filtrate was concentrated almost to dryness in a rotary evaporator (around 20 mmHg, temperature not exceeding 30° C.), the residue dissolved in 150 mL of water, stirred for 10 minutes at room temperature and filtered through a paper filter. Evaporation of water in a rotary evaporator followed by drying over P₂O₅ in a vacuum desiccator gave around 28 g of light-beige powder, which was hygroscopic.

### EXAMPLE 2

### Binding of Selected Pyridinium Salts to Sepharose Immobilized Heparin

Spectrophotometric methodologies were applied to estimate a degree of binding (DB) of the investigated compounds with heparin. This method is based on the absorption measurements of water solution of the investigated compound before and after a contact with heparin. The concentration was adjusted such that the absorbance in the region near the absorption maximum was around 1. The absorption spectra was collected for these prepared solutions. The same solutions of the investigated pyridinium salts were also incubated for 5 minutes with sepharose immobilized heparin (Heparin Sepharose CL-6B, Amersham Biosciences AB, Sweden) added in amount of 25 mg/mL. The suspensions of immobilized heparin with the analyzed compounds were then placed into Eppendorf microtubes and centrifuged at 13000 g for 4 minutes. The resulting clear solution over the sediment was introduced into a cuvette, and the absorption spectrum was measured. The degree of binding was estimated based on comparison of integrated area under the absorption curve for the solutions incubated and non-contacted with heparin.

DB was calculated according to the equation (1):

$$DB = \frac{IA - {IA}_{Hep}}{IA} \cdot 100 \% \qquad ( 1 )$$

where: DB=degree of binding,

IA=integrated area under absorption curve for sample without contact with immobilized heparin,

IA_{HEP}=integrated area under absorption curve for sample incubated with immobilized heparin.

The results of these experiments are shown below in Table A (Compounds 1 to 6 are cloride salts, compound 7 is a hydrochloride).

TABLE A
Binding of pyridinium salts to sepharose-immobilized heparin

| | | | Degree of |
|---|---|---|---|
| No. | Compound | Chemical structure | binding, [%] |
| 1 | 1-Methylnicotinamide(MNA) | 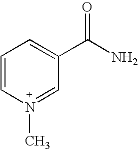 | >45 |
| 2 | 1,N′-Dimethylnicotinamide(MNA-Me1) | 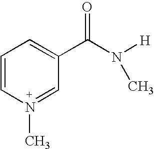 | 20-45 |
| 3 | 1-Methyl-N′,N′-diethylnicotin-amide (MNA-Et2) | 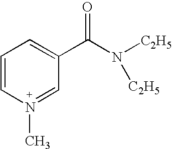 | 20-45 |
| 4 | 1-Methyl-N′-(hydroxymethyl)-nicotinamide (MNAF) | 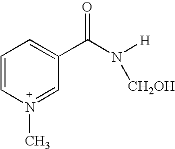 | 20-45 |
| 5 | 1-Methyl-3-acetylpyridine(MAP) | 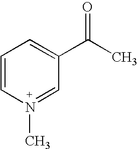 | 20-45 |
| 6 | 1-Propylnicotinamide(PNA) | 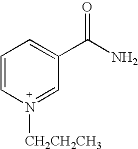 | 20-45 |
| 7 | Trigonelline(TRIG) | 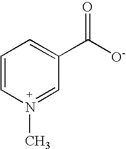 | <20 |
| 8 | Nicotinamide(NA) | 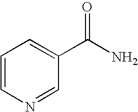 | <20 |

### EXAMPLE 3

### Preparation of Cream Containing a Composition of the Invention and Use of Cream in Clinical of Testing

| Component | Names of Ingredients | Concentration [%] |
|---|---|---|
| A | Polyglyceryl-2- | 6.00 |
| | Dipolyhydroxystearate, Dicaprylyl | |
| | Ether, Cocoglycerides, Cera Alba, | |
| | Sorbitan Sesquioleate, Aluminium | |
| | Stearates, Dicocoyl Pentaerythrityl | |
| | Distearyl Citrate | |
| B | Dicocoyl Pentaerythrityl Distearyl | 5.00 |
| | Citrate, Sorbitan Sesquioleate, Cera | |
| | Alba, Aluminium Stearates | |
| C | Glycerin | 5.00 |
| D | Ethylhexyl Stearate | 4.00 |
| E | Dicaprylyl Carbonate | 3.00 |
| F | Cocoglycerides | 2.00 |
| G | Tocopheryl Acetate | 1.00 |
| H | DMDM Hydantoin, Methylparaben, | 0.50 |
| | Phenoxyethanol, Propylparaben | |
| I* | 1-Methylnicotinamide Chloride, | 0.50 |
| | Wakame Extract | |
| J | Demineralized Water | 73.00 |

*Component I is a complex consisting of a 1:9 ratio of 1-methylnicotinamide chloride and wakame extract.

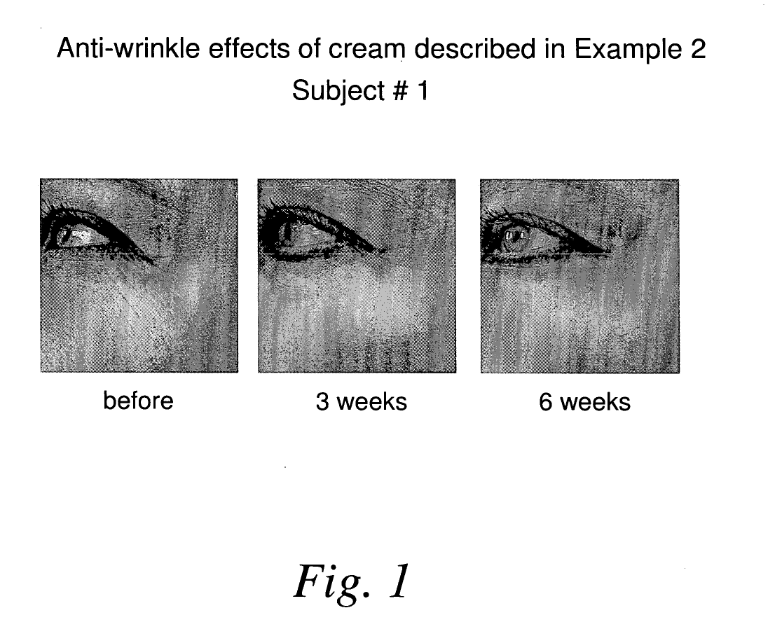

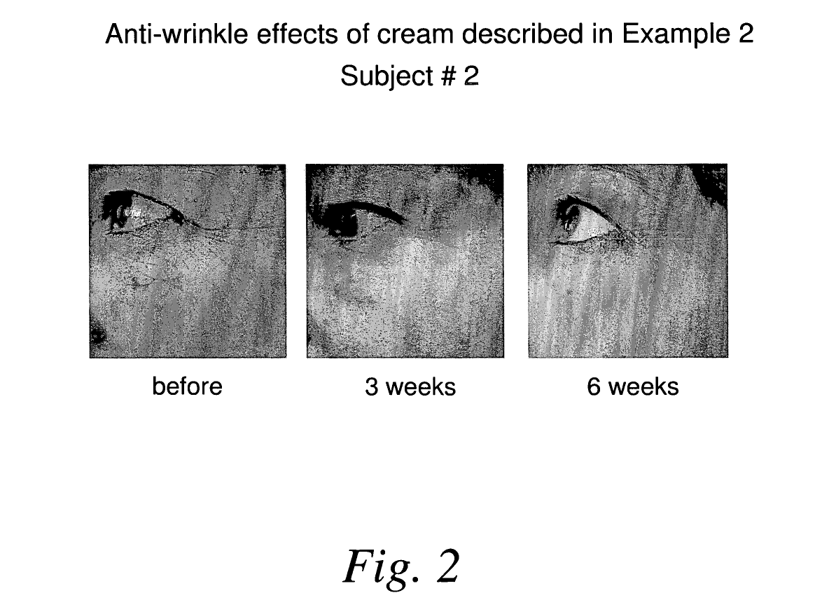

The cream described in Example 3 was tested in 15 women ages 38-81. The anti-aging efficacy of the cream, applied twice daily, was estimated at 3 and 6 weeks. All of the women were satisfied with the anti-aging effects of the cream, and no undesirable effects were noticed. Photographic documentation of these results are shown in FIGS. 1 and 2.

### EXAMPLE 4

### Additional Clinical Testing of the Cream Prepared in Example 3

The following tests were conducted in a manner that was consistent with the requirements of the Cosmetic Act of Mar. 30, 2001, with the following amendments:
effect of the cosmetic on human health (art. 11, section 1, item 4)
confirmation of product properties declared by the manufacturer (art. 11, section 1, item 6).

Test subjects participating in the study were recruited in conformity with the applicable European Community regulations (according to the provisions of the Helsinki Declaration of 1964 with subsequent amendments). Before the commencement of the tests, the subjects received no special instructions concerning the diet or lifestyle, as it was assumed that the cream from Example 3 should be tested under the conditions approximating as closely as possible the actual conditions of consumer use.

The group of subjects recruited for application tests and laboratory assessment included:
for greasing level measurements (measurement of sebum on skin surface): 15 subjects aged 38 to 55 with extremely dry and sensitive skin
for moisturizing level measurements: 27 subjects aged 39 to 56
for skin elasticity measurements: 25 subjects aged 32 to 56
for skin-smoothing effect of the cream: 20 subjects aged 30 to 56 with various skin types.

The face skin of the test subjects (women) demonstrated no pathologic changes at the beginning of the testing period.

### Testing Method

Measurements were conducted in 2 phases: the preliminary phase, which was an assessment of the greasing level in a 48-hour testing period, and the proper test phase, which was an assessment of the effect of the cream on the skin moisturizing level, elasticity and smoothness during a 28-day period of regular application.

Apparatus

Measurements were taken using Courage & Khazaka Visioscan VC/SELS software, and the following apparatus were used in the tests (see: http://www.courage-khazaka.de/):

Sebumeter—for measurements of greasing level by measuring the sebum on skin surface;

Corneometer—for measurements of skin moisturizing level by measuring any change in dielectric constant due to skin surface hydration (see, e.g., U. Heinrich, U. Koop, et al. International Journal of Cosmetic Science, 2003, 25, 45-51); and

Cutometer SEM 575—for measurements of skin elasticity by measuring the resistance of the skin to be sucked up by negative pressure (firmness) and its ability to return into its original position (elasticity)

### Testing Conditions

The assessments were carried out under the supervision of a licensed beautician and a doctor of pharmaceutical sciences. The relaxing time prior to measurements was 40 minutes. The measurements were carried out in an air-conditioned room with air temperature ranging from 19 to 23° C. and relative humidity ≧50%. For greasing level measurements, the air temperature was 19-23° C. and the relative humidity was 80%. For moisturizing level measurements, the air temp. was 20° C. and the relative humidity was 50%. For elasticity measurements, the air temp. was 19-23° C. and the relative humidity was 50%. For skin smoothness measurements, the air temp. 19-23° C. and the relative humidity 50%.

The preliminary and proper testing phase measurements were performed in female subjects having jobs and household duties, and in connection with such lifestyle frequently exposed to stress, which was reflected in their skin condition.

Before the commencement of the tests, the subjects received no special instructions concerning the diet or lifestyle, as it was determined that the cream should be tested under the conditions approximating as closely as possible the actual conditions of consumer use. Therefore, the measurement results were affected exclusively by such factors as: skin type, stress, fatigue and environmental conditions, etc.

### Testing Procedure

The subjects taking part in the laboratory tests were given the cream to use at home and were obliged to apply the cream on the measurement days in the laboratory, on the remaining days of the 4-week testing period at home. The measurements were conducted during the preliminary phase and the proper test phase.

The persons carrying out the measurements were obliged to observe and record the users' comments concerning reactions of the skin to the tested cream and collect detailed information from the test participants.

### Measurements

Preliminary Phase - Greasing level measurement Measurement areas on the subjects' foreheads, under the eyes, on the cheeks and neck were selected. The first measurement was performed before the application of the cream. The next measurements were performed 3, 5 and 48 hours after cream application. The measurement result was expressed as an arithmetic mean from 35 measurements. Such methodology minimizes the effect of intrinsic and extrinsic factors on the obtained results.

### Proper Test Phase

The first measurement of skin moisturizing level was performed in the selected areas: on the forehead, cheeks and neck, before the application of the cream. The next measurements were performed every other day for the period of 28 days. Results obtained on the particular days are expressed as arithmetic means from 27 measurements.

The first measurement of skin elasticity was performed before the application of the cream. The next measurements were performed at 7-day intervals for the period of 28 days. The results are expressed as arithmetic means from 3 measurements, i.e., measurements performed three times on each test participant. The measurement of skin smoothness was performed in the selected areas, at the sites where wrinkles are present (in most subjects on the upper part of the cheek, near the eye).

### Substantiation of the Testing Methodology

The purpose of the preliminary phase was to confirm, or to exclude, the effect of the cream on skin greasing level, and to determine the time of action. Confirmation of consistent greasing effect of the cream was the basis to undertake further tests. The purpose of the proper testing phase was to determine the effect of regular cream use on: skin moisturizing level, elasticity, smoothness, and change of skin condition.

### Results of Laboratory Measurements

Laboratory measurements demonstrated that the cream is characterized by a good and long-lasting greasing effect. As shown in Table 1, 48 h after application of the product, skin greasing is still maintained at the level equivalent to the greasing level of normal skin. As shown in Table 2, moisturizing level measurements demonstrated that the cream is characterized by a good and long-lasting moisturizing effect. After regular application of the cream for 4 weeks, skin moisturizing level increased, on the average, by 48%. Such high increase of moisture level indicates that the effect of the cream involved regulation of water balance in the skin. The skin of all test participants demonstrated moisture level typical of normal skin.

TABLE 1
RESULTS OF GREASING LEVEL MEASUREMENTS

| | BASELINE | RESULT | RESULT | RESULT |
|---|---|---|---|---|
| MEASUREMENT | (“0”) | AFTER | AFTER | AFTER |
| SITE | TEST | 3 h | 5 h | 48 h |
| Forehead | 68 | 89 | 93 | 93 |
| Cheeks | 36 | 56 | 55 | 57 |
| Under eyes | 34 | 50 | 54 | 54 |
| Neck | 28 | 39 | 40 | 37 |

TABLE 2
RESULTS OF MOISTURIZING LEVEL MEASUREMENTS

| | Day | | | | | | | | | | | | | | |
|---|---|---|---|---|---|---|---|---|---|---|---|---|---|---|---|
| | Test 0 | 1 | 2 | 3 | 4 | 5 | 6 | 7 | 8 | 9 | 10 | 11 | 12 | 13 | 14 |
| Forehead | 55 | 69 | 71 | 73 | 75 | 76 | 75 | 76 | 75 | 76 | 76 | 76 | 75 | 76 | 76 |
| Cheeks | 59 | 68 | 82 | 93 | 94 | 94 | 95 | 96 | 95 | 96 | 94 | 93 | 93 | 92 | 93 |
| Neck | 70 | 81 | 92 | 103 | 104 | 103 | 103 | 104 | 104 | 104 | 105 | 104 | 105 | 104 | 104 |

As shown in Tables 3 and 4, elasticity measurements demonstrated that regular application of the cream for 4 weeks improved skin elasticity to a large extent. An increase of all the measured parameters was noted:

Immediate distension to final deformation ratio (Ua/Uf)—the most sensitive skin elasticity parameter, increased by 51% during the 28-day trial period;

Immediate retraction to immediate distension ratio (Ur/Ue)—by 49%;

Viscoelasticity to immediate distention ratio (Uv/Ue)—by 46%; and

Immediate retraction to final deformation ratio (Ur/Uf)—by 49% Smoothness measurements demonstrated that regular application of the cream for 28 days results in good and long-lasting smoothening of the skin, correlated with the good moisturizing effect. The smoothness parameter increased by 48%, toughness was reduced by 42%, scaling was reduced by 41%, and anti-wrinkling effect increased by 40%.

TABLE 3
RESULTS OF ELASTICITY MEASUREMENTS

| | | | | | | % |
|---|---|---|---|---|---|---|
| | BASELINE | | | | | CHANGE |
| | (“0”) | | | | | AFTER 4 |
| PARAMETER | TEST | WEEK 1 | WEEK 2 | WEEK 3 | WEEK 4 | WEEKS |
| Ua/Uf | 0.406 | 0.431 | 0.485 | 0.599 | 0.614 | 51 |
| Ur/Ue | 0.449 | 0.499 | 0.599 | 0.615 | 0.669 | 49 |
| Uv/Ue | 0.410 | 0.389 | 0.365 | 0.260 | 0.221 | 46 |
| Ur/Uf | 0.483 | 0.577 | 0.618 | 0.697 | 0.719 | 49 |

TABLE 4
RESULTS OF TESTING WITH SELS METHOD

| | | | | | | % |
|---|---|---|---|---|---|---|
| | BASELINE | | | | | CHANGE |
| | (“0”) | | | | | AFTER 4 |
| PARAMETER | TEST | WEEK 1 | WEEK 2 | WEEK 3 | WEEK 4 | WEEKS |
| SE sm | 18.954 | 19.672 | 22.891 | 26.472 | 28.052 | 48 |
| SE r | 13.651 | 12.121 | 9.533 | 8.156 | 7.918 | 42 |
| SE sc | 8.387 | 7.938 | 6.187 | 5.485 | 4.948 | 41 |
| SE w | 19.673 | 18.895 | 16.307 | 14.566 | 11.804 | 40 |

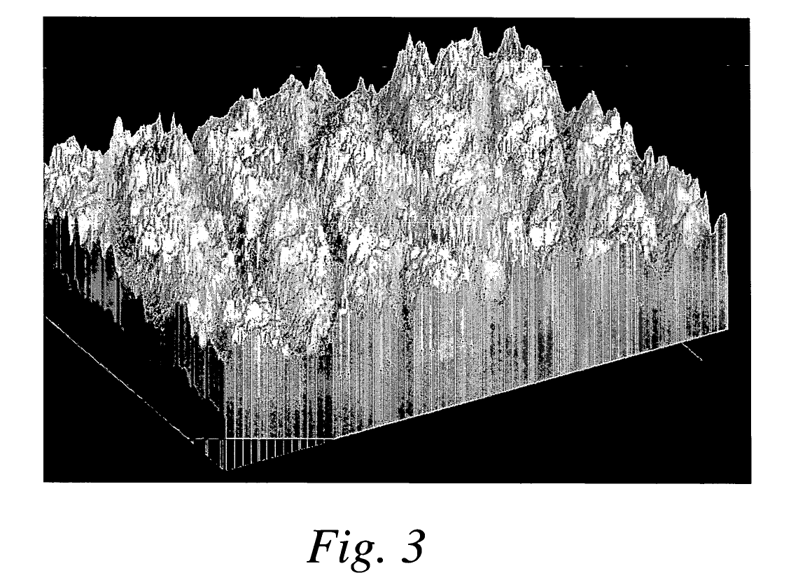

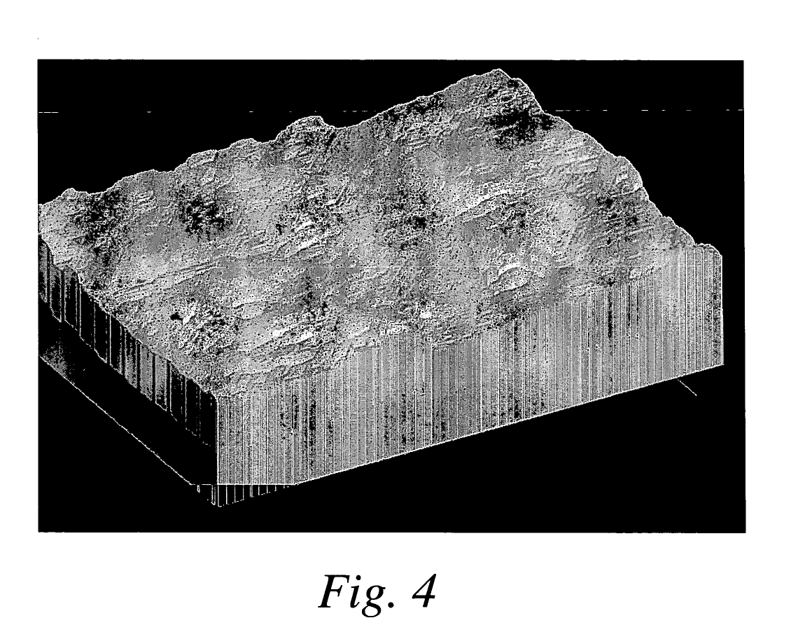

FIG. 3 demonstrates skin smoothness of before use of the cream, and FIG. 4 demonstrates skin smoothness after four weeks of cream use by a subject. These figures were obtained using SELS (Surface Evaluation of the Living Skin) software with a Visioscan VC 98 from Courage & Khazaka (see: http://www.courage-khazaka.de/products/p_vc_{—}98.htm).

1. A composition comprising wakame seaweed or wakame extract, and a compound of Formula I: 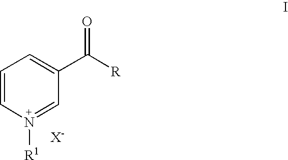
wherein
R represents the group NR²R³, OH, or C₁₋₄-alkyl;
R¹ and R² each, independently, represent hydrogen or C₁₋₄-alkyl;
R³ represents hydrogen, C₁₋₄-alkyl, C₁₋₄-alkyl-OH, or C₁₋₄-alkoxy; and
X⁻ is a physiologically suitable counter-anion.

2. The composition of claim 1 in which R represents the group NR²R³.

3. The composition of claim 1 in which R² represents methyl, ethyl, or hydrogen.

4. The composition of claim 1 in which R³ represents CH₂OH, ethyl, or hydrogen.

5. The composition of claim 1 in which R represents OH, NH₂, N(H)CH₃, N(Et)₂, N(H)CH₂0H or CH₃.

6. The composition of claim 1 in which R¹ represents methyl, ethyl or propyl.

7. The composition of claim 1 wherein the compound of Formula I is selected from a 1-methylnicotinamide salt, 1,N′-dimethylnicotinamide salt, 1-methyl-N′,N′-diethylnicotinamide salt, 1-methyl-N′-(hydroxymethyl)-nicotinamide salt, 1-methyl-3-acetylpyridine salt, 1-propylnicotinamide salt, trigonelline salt, and nicotinamide.

8. The composition of claim 1 wherein the compound of Formula I is a 1-methylnicotinamide salt.

9. The composition of claim 1 wherein X⁻ is chloride, benzoate, salicylate, acetate, citrate or lactate.

10. The composition of claim 1, wherein the compound of Formula I is present in said composition in a concentration of between 0.001% and 30% by weight of said composition.

11. A composition comprising a glycosaminoglycan (GAG) and a compound of Formula I: 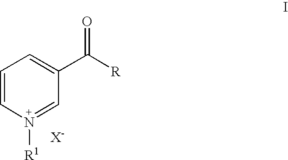
wherein
R represents the group NR²R³, OH, or C₁₋₄-alkyl;
R¹ and R² each, independently, represent hydrogen or C₁₋₄-alkyl;
R³ represents hydrogen, C₁₋₄-alkyl, C₁₋₄-alkyl-OH, or C₁₋₄-alkoxy; and
X⁻ is a physiologically suitable counter-anion.

12. The composition of claim 11 in which R² represents methyl, ethyl, or hydrogen.

13. The composition of claim 11 in which R³ represents CH₂OH, ethyl, or hydrogen.

14. The composition of claim 11 in which R represents OH, NH₂, N(H)CH₃, N(Et)₂, N(H)CH₂OH or CH₃.

15. The composition of claim 11 in which R¹ represents methyl, ethyl or propyl.

16. The composition of claim 11 wherein X⁻ is chloride, benzoate, salicylate, acetate, citrate or lactate.

17. The composition of claim 11 wherein the compound of Formula I is selected from a 1-methylnicotinamide salt, 1,N′-dimethylnicotinamide salt, 1-methyl-N′,N′-diethylnicotinamide salt, 1-methyl-N′-(hydroxymethyl)-nicotinamide salt, 1-methyl-3-acetylpyridine salt, 1-propylnicotinamide salt, trigonelline salt, and nicotinamide.

18. The composition of claim 11, wherein the compound of Formula I is a 1-methylnicotinamide salt.

19. The composition of claim 11, wherein the glycosaminoglycan (GAG) is heparin, heparin sulfate, keratan sulfate, dermatin, dermatin sulfate, heparin-hyaluronic acid, chondroitin, chondroitin sulfate (e.g., chondroitin 6-sulfate and chondroitin 4-sulfate), chitin, chitosan, acetyl-glucosamine, hyaluronic acid, aggrecan, decorin, biglycan, fibromodulin or lumican, or combinations thereof.

20. The composition of claim 11, wherein the glycosaminoglycan (GAG) is heparin.

21. The composition of claim 11, wherein the source of the glycosaminoglycan is IS wakame seaweed or wakame seaweed extract.

22. A method of treating skin diseases and disorders in a subject in need thereof by administering to the subject a composition comprising wakame seaweed or wakame extract, and a compound of Formula I.

23. A method of treating skin diseases and disorders in a subject in need thereof by administering to the subject a composition comprising a glycosaminoglycan (GAG) and a compound of Formula I.

24. The method of claim 22, wherein the skin diseases or disorders are selected from the group consisting of sunburn, bums, scalds, skin wounds, wrinkles, oxidative damage in the skin and UV-induced skin damage.

25. A method of restoring firmness and tonicity to the skin in a subject in need thereof by administering to the subject a composition comprising wakame seaweed or wakame extract, and a compound of Formula I.
